(19) United States
(12) Patent Application Publication
(10) Pub. No.: US 2010/0114327 A1
(43) Pub. Date: May 6, 2010

(54) VALVE

(75) Inventor: Gabriel SOBRINO-SERRANO, Kinvara (IE)

Correspondence Address: CHOATE, HALL & STEWART LLP, TWO INTERNATIONAL PLACE, BOSTON, MA, 02110, US

(73) Assignee: Vysera Biomedical Limited, Galway (IE)

(21) Appl. No.: 12/488,034

(22) Filed: Jun. 19, 2009

Related U.S. Application Data
(60) Provisional application No. 61/074,403, filed on Jun. 20, 2008.

(51) Int. Cl. A61F 2/04

(52) U.S. Cl. 623/23.68

(57) ABSTRACT

An esophageal valve has a central restriction to restrict flow through the valve. The valve has a proximal guide to guide antegrade flow to the restriction and a distal guide to guide retrograde flow to the restriction.

### CROSS-REFERENCES TO RELATED APPLICATIONS

The present application claims the benefit of U.S. Provisional Application No. 61/074,403 filed Jun. 20, 2008, the complete contents of which is incorporated herein by reference.

### INTRODUCTION

The invention relates to devices useful in the treatment of GERD (Gastro Esophageal Reflux Disease)

GERD is often caused by a failure of the anti-reflux mechanism. In healthy patients the anti-reflux mechanism is comprised of the lower esophageal sphincter (LES) and the “Angle of His,” the angle at which the esophagus enters the stomach. These create a valve that prevents duodenal bile, enzymes, and stomach acid from traveling back into the esophagus where they can cause burning and inflammation of sensitive esophageal tissue.

A surgical treatment, which is often preferred over longtime use of medication, is the Nissen fundoplication. In this procedure the upper part of the stomach is wrapped around the LES (lower esophageal sphincter) to strengthen the sphincter and prevent acid reflux and to repair a hiatal hernia. The procedure is usually carried out laparoscopically.

In 2000 the U.S. Food and Drug Administration (FDA) approved two endoscopic devices to treat chronic heartburn. In one system stitches are put in the LES to create pleats to strengthen the muscle. Another, the Stretta Procedure, uses electrodes to apply radio frequency energy to the LES. The long-term outcomes of both procedures compared to a Nissen fundoplication are still being determined.

The NDO Surgical Plicator is also used for endoscopic GERD treatment. The Plicator creates a plication, or fold, of tissue near the gastroesophageal junction, and fixates the plication with a suture-based implant. The Plicator is currently marketed by NDO Surgical, Inc.

### SUMMARY OF THE INVENTION

According to the invention there is provided an esophageal valve having a central restriction to restrict flow through the valve, a proximal guide to guide antegrade flow to the restriction and a distal guide to guide retrograde flow to the restriction.

In one embodiment the proximal guide comprises a proximally open cup.

In one embodiment the distal guide comprises a distally open cup.

In one case the valve is of generally hourglass shape.

The valve may comprise a frame. In one case the frame comprises a plurality of ribs.

In one embodiment the valve comprises a body supported by the frame.

In one case the frame comprises stiff ribs and the body comprises a flexible material.

The body may be overmoulded onto the frame.

In one embodiment the frame comprises at least three ribs.

In one case the frame comprises three ribs.

In other cases the frame comprises four or five ribs.

In one embodiment the ribs extend along the valve at least partially between a distal end and a proximal end of the valve.

At least some of the ribs may extend along the valve restriction.

In one case the ribs are radially movable in the region of the restriction.

The ribs may be movable from a normally constricted configuration to an enlarged configuration to facilitate in response to antegrade flow and/or retrograde flow through the valve. The ribs can return to the normally constricted configuration.

In one embodiment the ribs comprise fibers.

In one case the valve comprises a flap which is movable from an open to a closed configuration. The flap may be located at the restriction.

There may be a support structure for the valve. In one case the support structure comprises a luminal prosthesis such as a stent.

In one embodiment the support structure comprises a scaffold to which the valve is mounted and the scaffold with the mounted valve are adapted to engage with a predeployed luminal prosthesis.

### BRIEF DESCRIPTION OF THE DRAWINGS

The invention will be more clearly understood from the following description thereof given by way of example only, in which:

FIG. 1 is an isometric view of a valve according to the invention;

FIG. 2 is an isometric view of a support stent with the valve of FIG. 1 mounted therein;

FIG. 3 is an isometric view of the valve of FIG. 1;

FIG. 4 is a plan view of the valve of FIG. 5;

FIG. 5 is a side elevational view of the valve of FIG. 5;

FIG. 6 is an isometric view of a stent incorporating a valve of;

FIGS. 7 and 8 are plan views of the stent and valve of FIG. 6;

FIG. 9 is a cut-away view of the valve and stent of FIGS. 6 to 8;

FIGS. 10 and 11 are respectively side elevational and plan views of another valve of the invention which has four ribs;

FIG. 12 is a cut-away view of another valve in which the valve has ribs and openings;

FIG. 13 is an isometric view of the valve of FIG. 12 in a support structure;

FIG. 14 is an isometric cut-away view of the valve of FIGS. 12 and 13;

FIG. 15 is an elevational view of a valve with six ribs;

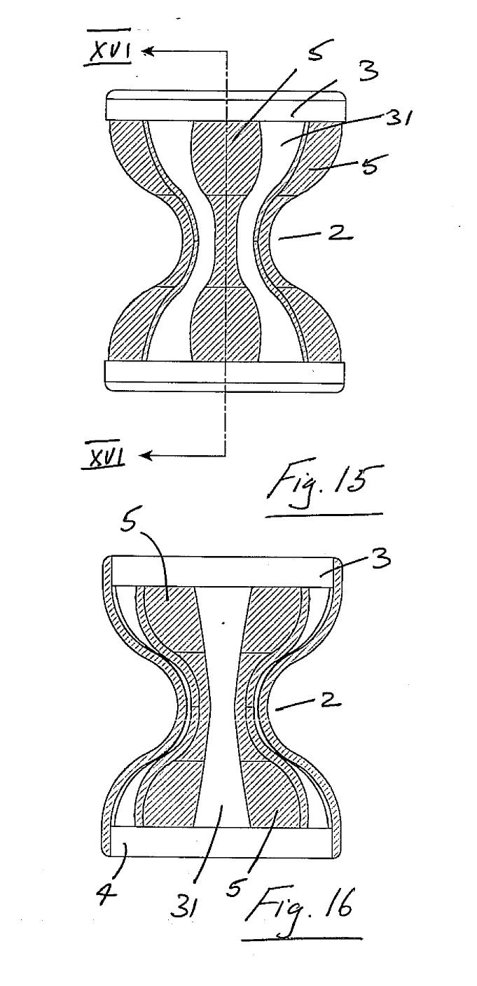

FIG. 16 is a cross sectional view on the line XVI to XVI in FIG. 15;

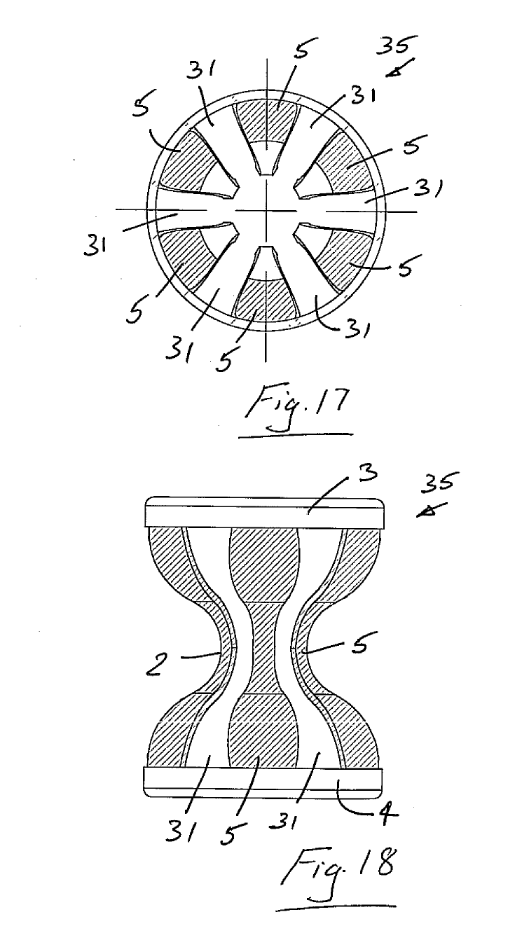

FIG. 17 is a plan view of the valve of FIGS. 16 and 17;

FIG. 18 is an elevational view of the valve of FIG. 17;

FIG. 19 is an elevational view of another valve of the invention with interconnected ribs;

FIG. 20 is a cut-away isometric view of the valve of FIG. 19;

FIG. 21 is an elevational view of a valve;

FIG. 22 is a cross sectional view on the line XXII to XXII of FIG. 21; and

FIGS. 23 to 26 are cut-away views of a support structure with a valve having a centrally located plug or flap;

### DETAILED DESCRIPTION

This invention is a prosthetic esophageal valve, which prevents reflux from entering into the esophagus. The valve is specifically designed to allow swallowed food to pass distally through it and also to allow retrograde flow of liquid and gasses proximally during belching or vomiting.

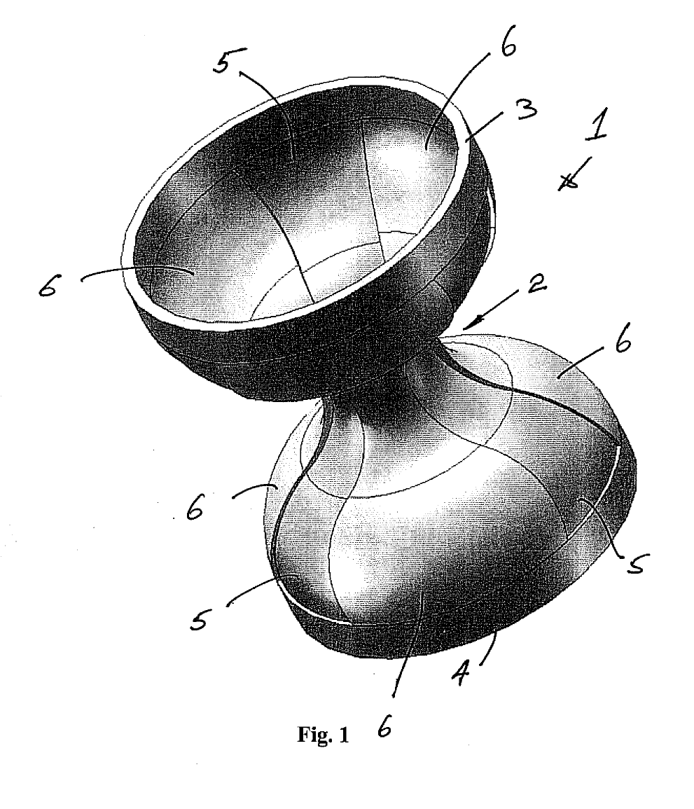

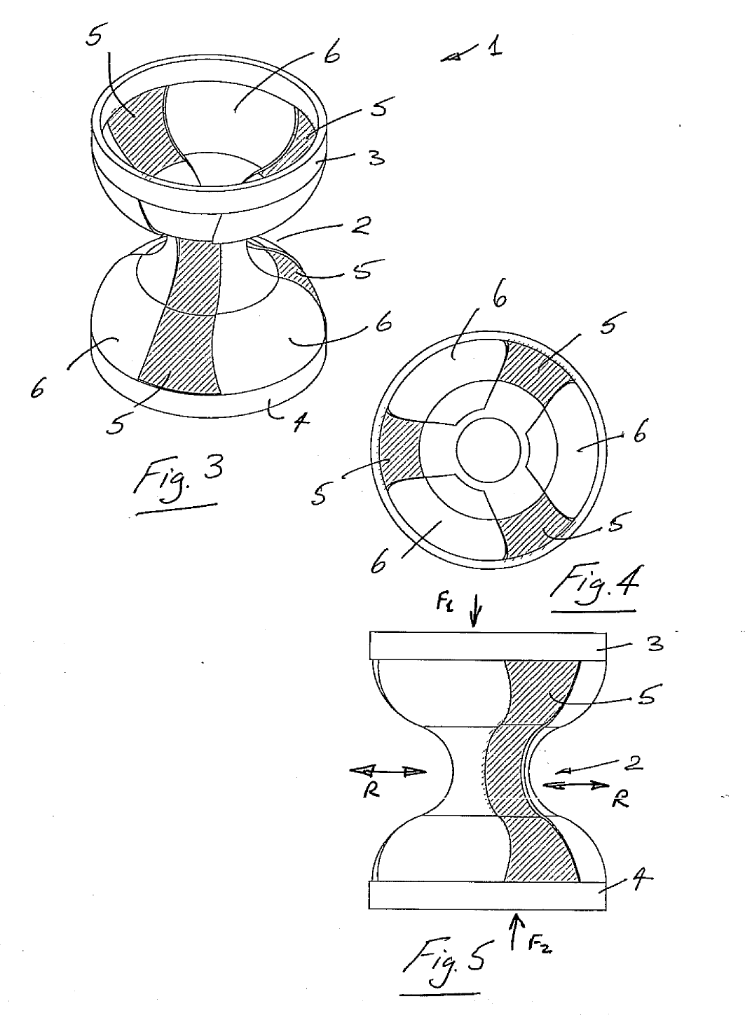

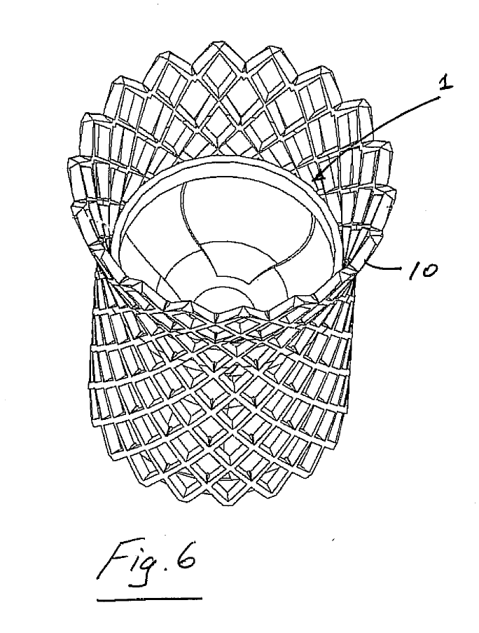

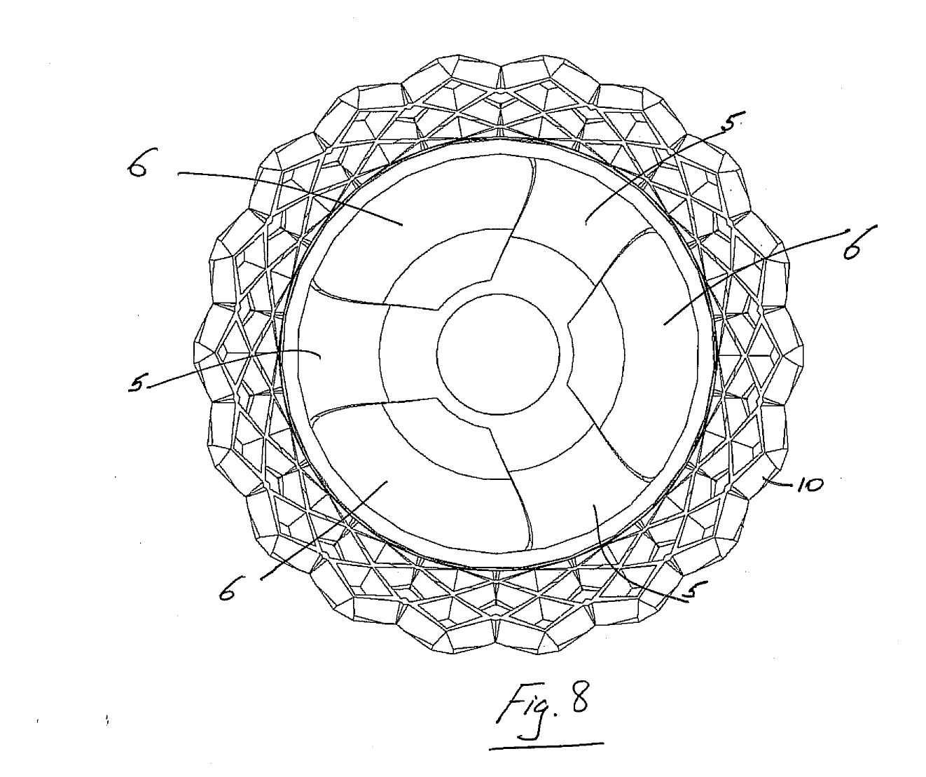

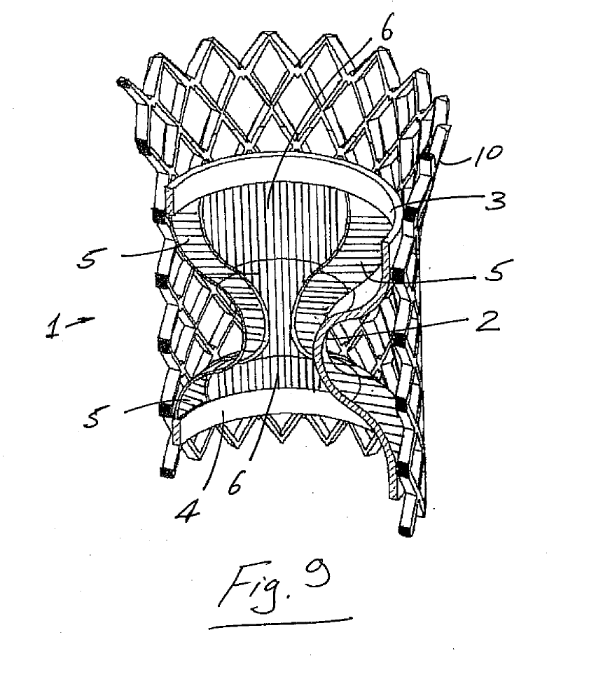

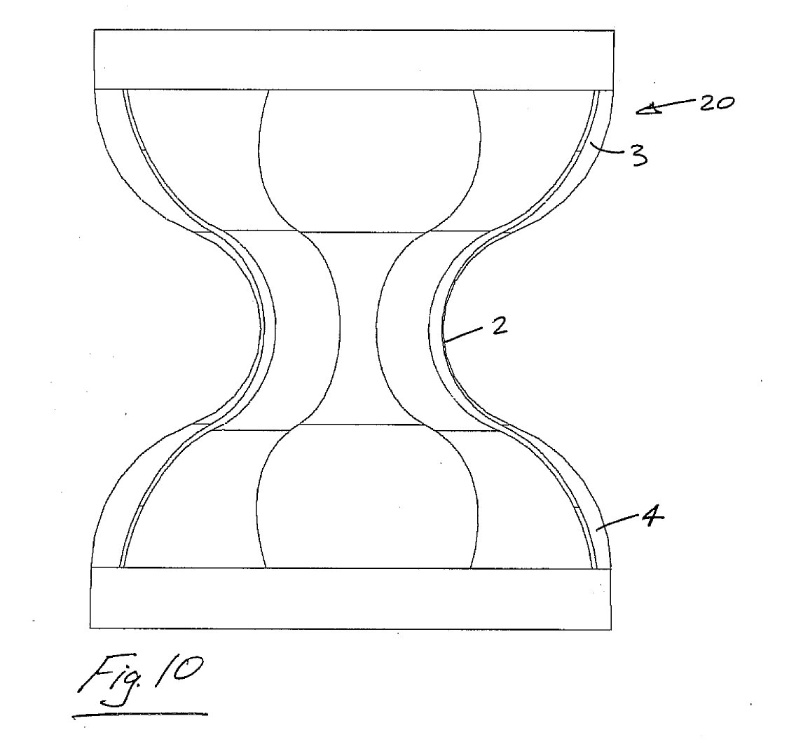

Referring to the drawings and initially to FIGS. 1 to 9 there is illustrated an esophageal valve 1 having a central restriction 2 to restrict flow through the valve. The valve 1 has a proximal guide which in this case is in the form of a proximally opening cup 3. The valve 1 also has a distal guide which in this case is in the form of a distally open cup 4. The valve 1 in this instance is of generally hourglass shape in which both ends are conically shaped.

The valve may be of any suitable biocompatible materials. It may be of a biocompatible polymeric material having properties which allow the valve to function as described.

The materials used for the production of this valve should have a % elongation between 50% and 3000%. The material should also have a tensile strength of between 0.01 and 5 N/mm² Additionally the material could have an antimicrobial action to prevent colonisation when in-vivo. Additionally the material can be elastic or viscoelastic and can optionally be an open cell foam. The density of the material should be between 0.1 g/cm3 to 1.5 g/cm3.

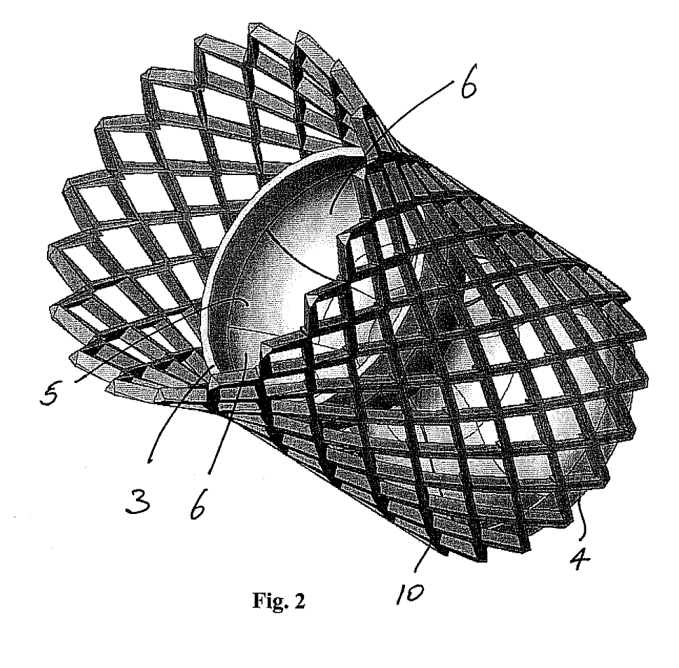

The valve may be housed within a soft scaffold or stent 10 to provide additional support as shown in FIGS. 2 and 6 to 9.

A support such as a stent may be deployed at a suitable location and the valve subsequently delivered into the support. Alternatively the valve may have a support and the valve and support scaffold are delivered and deployed together at a desired location. In another version the valve is mounted to a support scaffold and the valve and scaffold are delivered and deployed to a pre-deployed support stent.

The valve may be of a material that can be collapsed into a reduced diameter delivery configuration. On deployment, the valve expands or is expandable to the Normal enlarged deployed configuration.

The conical sections 3, 4 act as a means to direct food through the lumen rather than around the outside of the device whereas the narrow restriction 2 in the middle of the valve functions as a means of preventing flow.

The force provided by the valve to restrict flow is dependent on a number of factors including:
the degree of restriction in the centre of the valve;
the angle or curvature between the end of the device and the central restriction;
whether the angle (or curvature) of the proximal and distal ends of the device are the same. Different angles will create different degrees of restriction in the proximal and distal directions;
the ‘pass through’ force can also be manipulated by changing the material properties from which the valve is made; and
the properties of the valve will also be influenced by the external forces placed upon it by the natural movement and pressure of the esophagus. This external force may augment the functioning of the valve by contributing to the degree of restriction.

The valve can incorporate longitudinally alternating materials as illustrated. These materials can be alternately stiff and elastic or of varying degrees of stiffness/elasticity. There may be relatively ribs 5 and soft segments 6. The soft material may be overmoulded over the stiff ribs 5. In this case the valve has three longitudinally extending ribs.

The angle that the proximal end 3 of the device makes with the restriction 2 should be between 10 and 80° to the horizontal.

The luminal diameter of the restriction 2 in the centre of the valve can be between 0 and 20 mm.

The elongation of the material used to construct the restriction can be between 50 and 3000%.

The tensile strength of the material used to construct the restriction can be between 0.01 and 5 N/mm2.

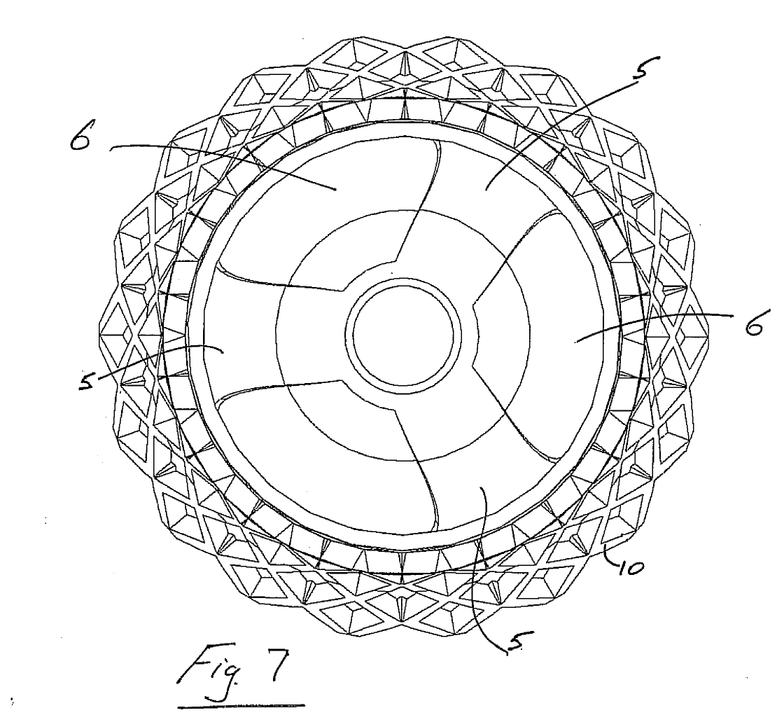

In order to facilitate the normal passage of food in the esophagus the restriction is capable of opening, in the proximal direction, with an axial force of between 50 and 100 g. Such antegrade flow is indicated by F₁ in FIG. 5.

In order to facilitate normal belching and vomiting the restriction is capable of opening, in the distal direction, with a pressure of between 200 and 500 mmH₂O. Such retrograde flow is indicated by F₂ in FIG. 5.

This design creates the optimum valve conditions while positioned in the lumen of the esophagus. Previous approaches have required devices to be placed either on the outside of the esophagus or under the mucosal surface tissue of the esophagus. Thus the current invention circumvents the need for invasive procedures.

The valve allows retrograde opening at precisely defined pressures. Because the valve does not invert to facilitate belching and vomiting it does not have to reorient itself periodically.

Many variations of valve can be produced to treat varying severity of disease.

The device can also be made in a very low profile fashion to facilitate its crimping and subsequent delivery.

The valve of the invention requires very low actuation pressure. It can function asymmetrically to mimic the various bodily functions in the esophagus. The valve may have a very low profile to enable crimping.

The valve may have any desired number of ribs 5 to create forces to control the recovery of the valve into the working position after pressure is released. Some of the ribs form a frame that can be made of a material with a significantly lower elasticity. This frame can be overmolded with another material, which occupies the spaces between frames ribs. This second material has a higher elongation that the frame.

The purpose of the frame is to limit the elongongation of the valve in the axial direction while simultaneously allowing the valve to expand radially as illustrated by the arrows R in FIG. 5 to open the restriction 2. The ribs/frame 5 are inherently biased to return to radially contract the restriction 2.

The mechanism of the valve opening occurs when outward radial force causes radial circumferential expansion of the second overmolded material occupying the space between the frame ribs 5.

The number of ribs 5 in the frame can be three or higher which influences the force required to open the valve because of the quantity of elastic material occupying the overmolded gaps between the frame ribs.

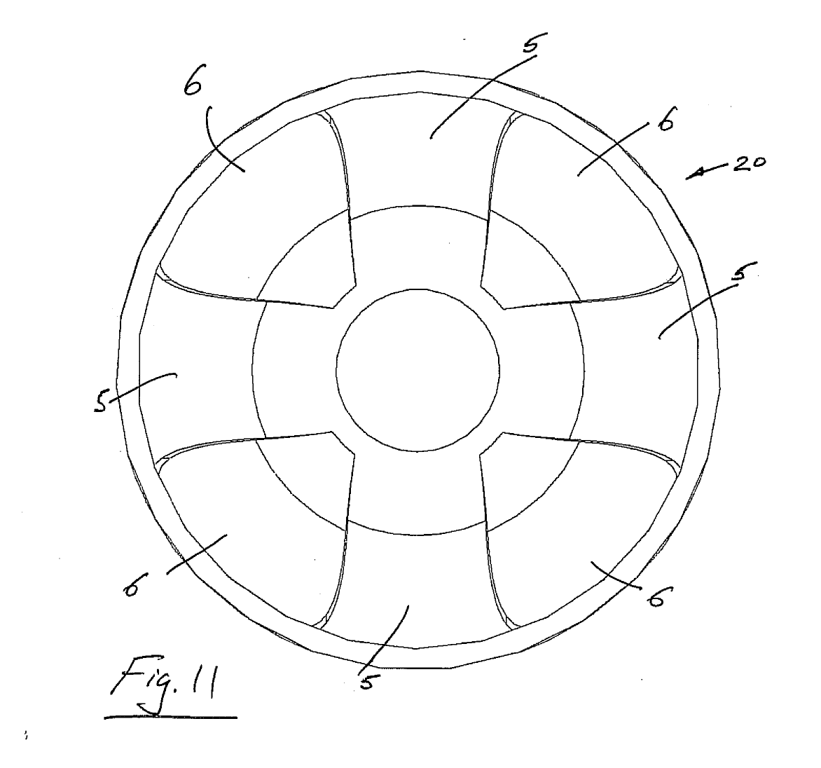

Referring to FIGS. 10 and 11 there is illustrated another valve 20 which is similar to the valve of FIGS. 1 to 9. In this case the valve has four ribs 5.

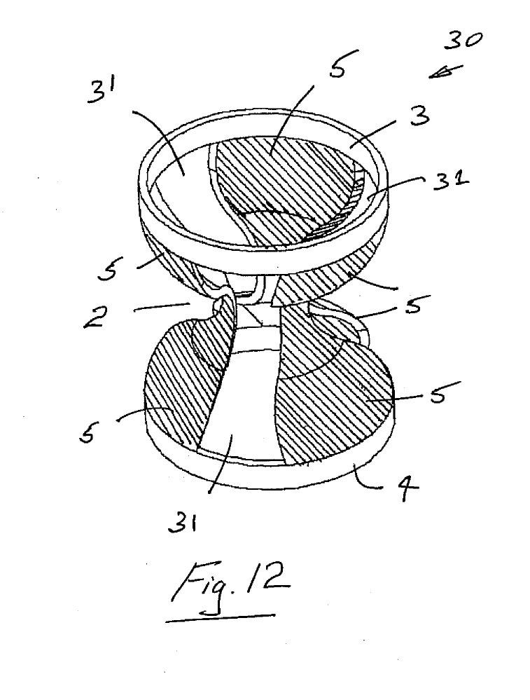

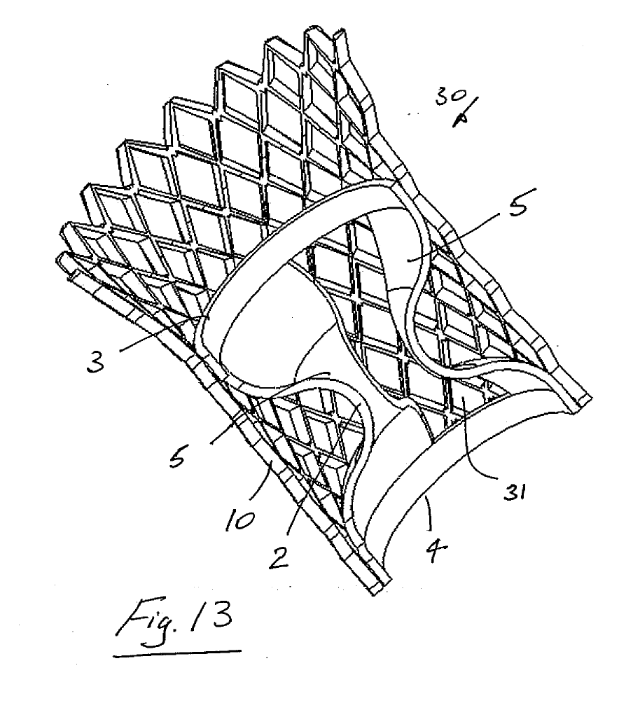

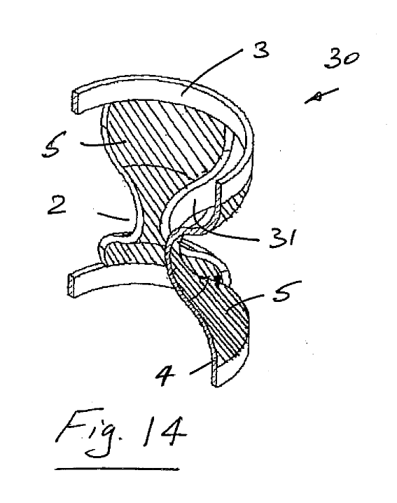

In an alternative embodiment illustrated in FIGS. 12 to 14 a valve 30 may have alternating ribs 5 and spaces 31. The spaces 31 may be overmolded with an additional continuous layer of material. Alternatively, the device may have a sleeve positioned within the central lumen. In this case the ribs act as a spring holding the sleeve closed.

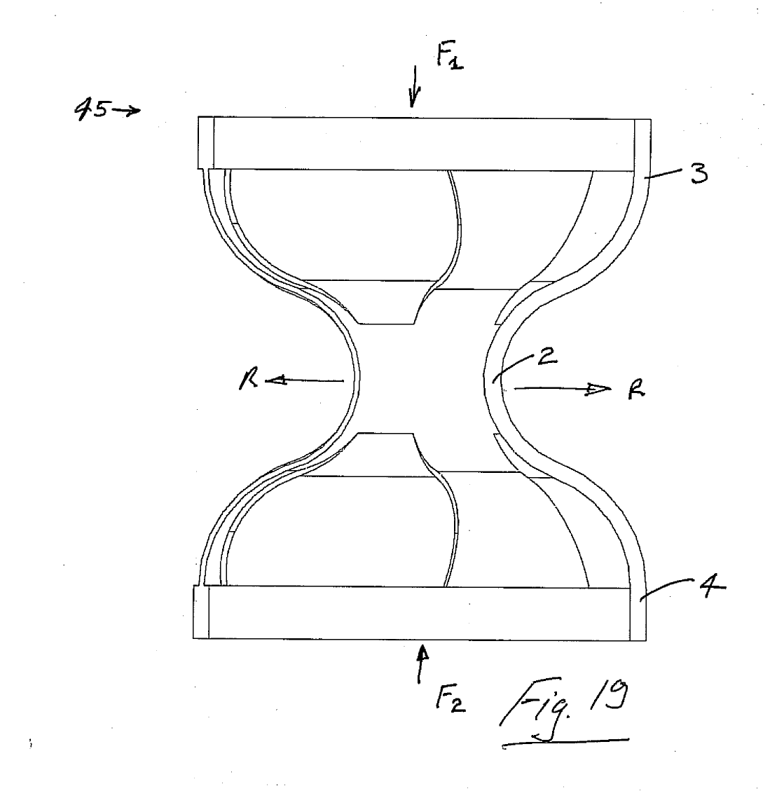

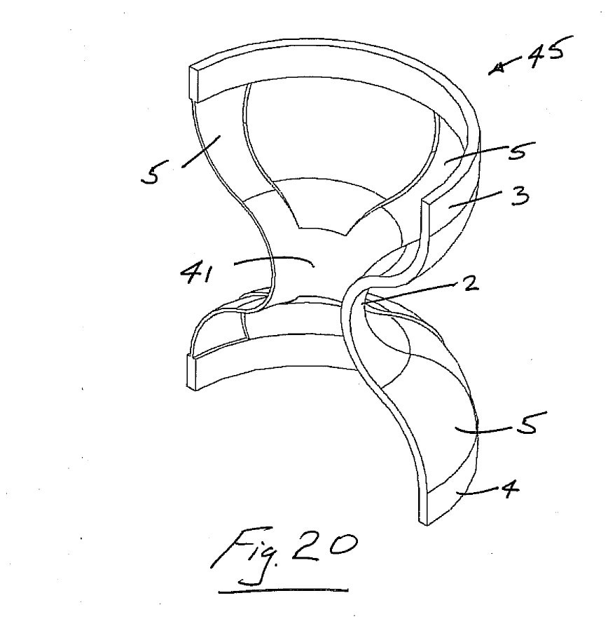

Another valve 40 is illustrated in FIGS. 17 to 18 which is similar to the valve of FIGS. 12 to 14. In this case the valve has six longitudinally extending thin ribs 5.

In fact, the ribs can also be pre-fabricated fibers, which are assembled such that they take the curved shape incorporating the restriction 2. The fibers can then be overmolded with an elastic material to hold them in their assembled configuration.

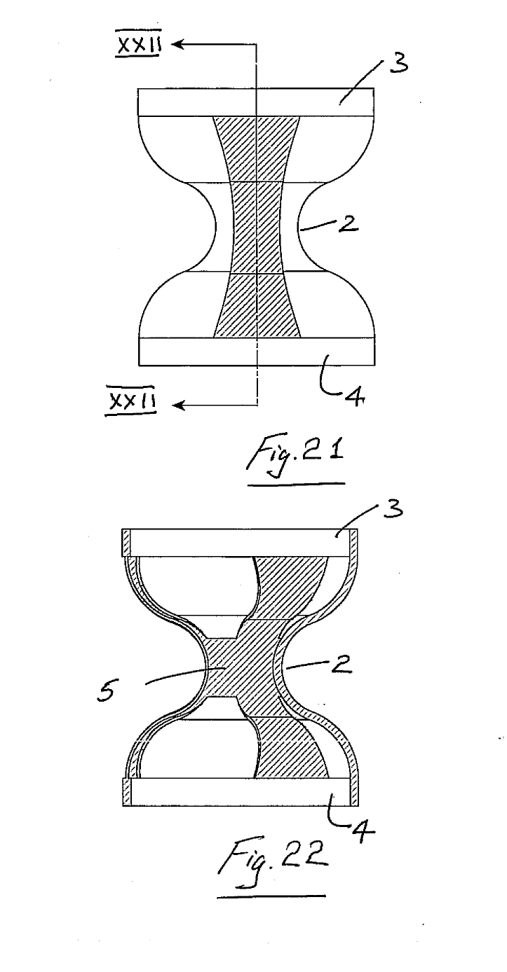

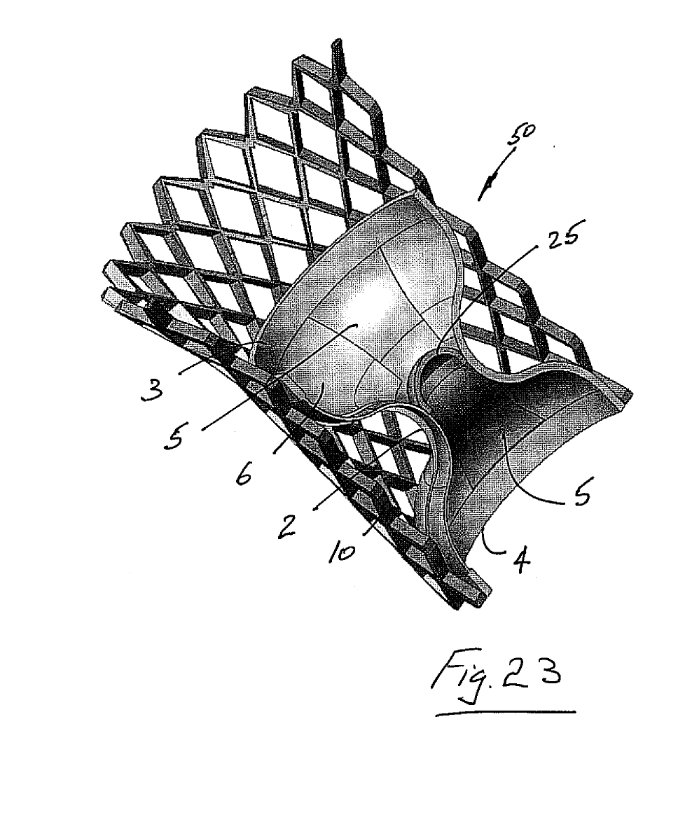

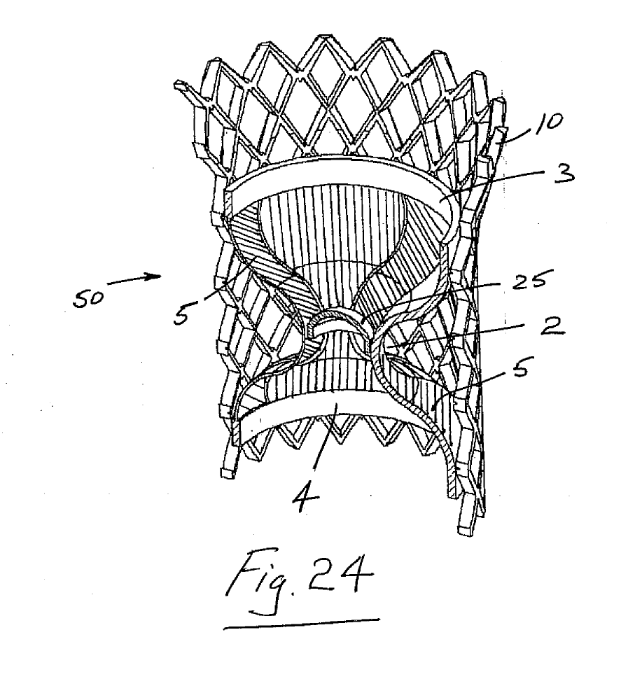

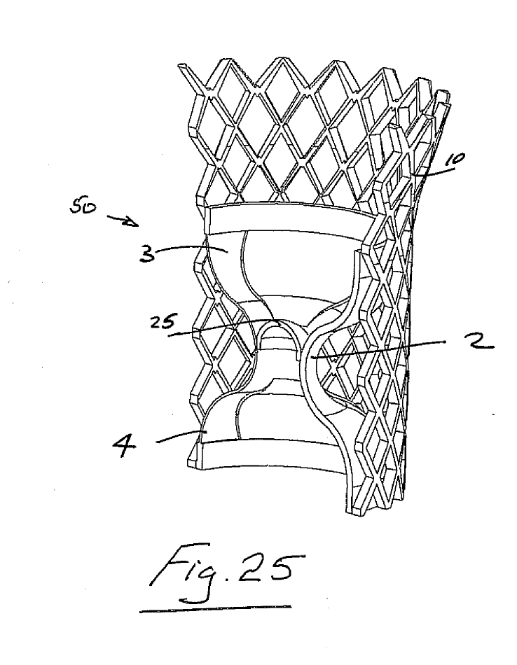

Referring to FIGS. 19 to 22 there is illustrated another valve 45 which in this case has ribs 5 which are interconnected, for example by a central connecting region 41 which defines a central sleeve. In this case the ribs 5 act as a spring to hold the central region 41 closed. The central region can be arranged to take account of the mechanical forces required.

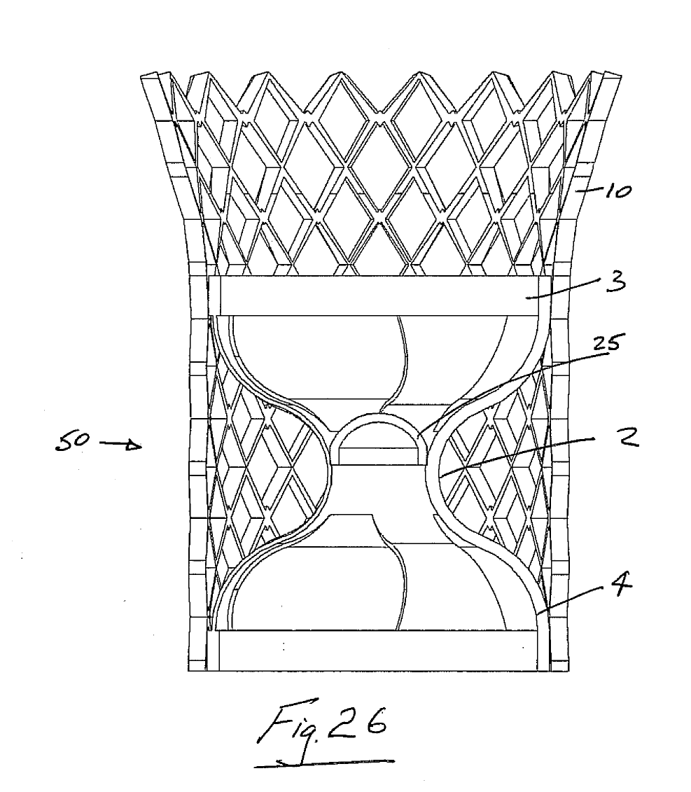

Referring to FIGS. 23 to 26 any of the valves of the invention such as a valve 50 may include a soft plug or flap valve 25 that in the closed lumen of the valve but which is capable of being displaced easily by the force of food being swallowed.

The valve of the invention may be used to treat gastro esophageal reflux disease.

The invention is not limited to the embodiments hereinbefore described, which may be varied in detail.

1. An esophageal valve having a central restriction to restrict flow through the valve, a proximal guide to guide antegrade flow to the restriction and a distal guide to guide retrograde flow to the restriction.

2. A valve as claimed in claim 1 wherein the proximal guide comprises a proximally open cup.

3. A valve as claimed in claim 1 wherein the distal guide comprises a distally open cup.

4. A valve as claimed in claim 1 wherein the valve is of generally hourglass shape

5. A valve as claimed in claim 1 wherein the valve comprises a frame.

6. A valve as claimed in claim 5 wherein the frame comprises a plurality of ribs.

7. A valve as claimed in claim 5 comprising a body supported by the frame.

8. A valve as claimed in claim 7 wherein the frame comprises stiff ribs and the body comprises a flexible material.

9. A valve as claimed in claim 7 wherein the body is overmoulded onto the frame.

10. A valve as claimed in claim 5 wherein the frame comprises at least three ribs.

11. A valve as claimed in claim 5 wherein the frame comprises three ribs.

12. A valve as claimed in claim 5 wherein the frame comprises four or five ribs.

13. A valve as claimed in claim 6 wherein the ribs extend along the valve at least partially between a distal end and a proximal end of the valve.

14. A valve as claimed in claim 13 wherein at least some of the ribs extend along the valve restriction.

15. A valve as claimed in claim 14 wherein the ribs are radially movable in the region of the restriction.

16. A valve as claimed in claim 15 wherein the ribs are movable from a normally constricted configuration to an enlarged configuration to facilitate in response to antegrade flow and/or retrograde flow through the valve.

17. A valve as claimed in claim 16 wherein the ribs return to the normally constricted configuration.

18. A valve as claimed in claim 6 wherein the ribs comprise fibers.

19. A valve as claimed in claim 1 wherein the valve comprises a flap which is movable from an open to a closed configuration.

20. A valve as claimed in claim 19 wherein the flap is located at the restriction.

21. A valve as claimed in claim 1 comprising a support structure for the valve.

22. A valve as claimed in claim 21 wherein the support structure comprises a luminal prosthesis such as a stent.

23. A valve as claimed in claim 21 wherein the support structure comprises a scaffold to which the valve is mounted and the scaffold with the mounted valve are adapted to engage with a predeployed luminal prosthesis.
